(19) United States
(12) Patent Application Publication
(10) Pub. No.: US 2025/0221711 A1
(43) Pub. Date: Jul. 10, 2025

(54) DEVICES AND METHODS FOR FORMING AN ANASTOMOSIS BETWEEN TWO LUMENS

(71) Applicant: The Regents of The University of California, Oakland, CA (US)

(72) Inventors: Clifford Pereira, Oakland, CA (US); Gavin Pereira, Oakland, CA (US); Jonathon Schofield, Oakland, CA (US)

(21) Appl. No.: 18/705,337

(22) PCT Filed: Oct. 27, 2022
(86) PCT No.: PCT/US2022/048066

§ 371 Date: Apr. 26, 2024

Related U.S. Application Data
(60) Provisional application No. 63/273,647, filed on Oct. 29, 2021.

(51) Int. Cl. A61B 17/115; A61B 17/00; A61B 17/064; A61B 17/11

(52) U.S. Cl. CPC A61B 17/1152; A61B 17/064; A61B 2017/00876; A61B 2017/0647; A61B 2017/1107; A61B 2017/1132; A61B 2217/002

(57) ABSTRACT

A device for forming an anastomosis between two lumens may include a staple receiving assembly and a staple dispensing assembly. The staple receiving assembly may include a first body defining a bottom surface surrounding a first central opening and staple receiving cavities. The staple dispensing assembly may include a second body defining a top surface surrounding a second central opening and staple dispensing cavities. The device may also include a plurality of linear staples positioned within the staple dispensing cavities. The is designed to retain everted ends of the first and second lumens between the bottom surface and the top surface. Further, the staple dispensing assembly may propel the linear staples from the staple dispensing cavities through the retained everted ends of the first and second lumens toward the staple receiving cavities in order to form an anastomosis between the first lumen and the second lumen.

### BACKGROUND

This application claims priority to U.S. Provisional Application No. 63/273,647, filed Oct. 29, 2021, the disclosure of which is hereby incorporated by reference in its entirety for all purposes.

An anastomosis is a connection between two things, for example two lumens. A vascular anastomosis (VA) is a procedure whereby two ends of a severed blood vessel, such as an artery or vein, are joined in order to establish blood flow into or out of an organ or limb. Vascular anastomosis may be currently performed by hand sewing the two ends of the blood vessel together using needle and sutures, for example as shown in FIGS. 1A and 1B. A hand-sewn anastomosis can take roughly 45-60 minutes even for an experienced surgeon to complete, and can take longer for inexperienced surgeons or difficult-to-reach anatomic locations. The duration of hand sewing therefore has a long time that the tissue is denied blood (i.e. TTA), which is detrimental to the tissue health.

Existing devices for forming a vascular anastomosis include a device for example as shown in FIGS. 2A-2C, marketed as the Synovis® MCA microvascular coupling device. The device as shown in FIGS. 2A and 2B includes two rings with spikes, wherein ends of blood vessels are everted over the spikes of the rings. The rings are brought together and receive the spikes of the opposing ring, thereby coupling the ends of the blood vessels therebetween, wherein the rigid rings remain a part of the anastomosis after the procedure is performed. The device of FIGS. 2A-2C is disadvantageous in that the procedure has a TTA of 20-100 minutes, requires a high level of surgical dexterity to evert the blood vessels over the spikes, cannot be adapted for vessels over 5 mm diameter, and due to the rigid rings does not allow for expansion of the blood vessel with every heartbeat.

Accordingly, there is a need for a device for forming an anastomosis, for example a vascular anastomosis, with a low TTA, not requiring a high level of surgical dexterity, adaptable to vessels over 5 mm in diameter and/or allowing for expansion of the blood vessel with every heartbeat.

### BRIEF SUMMARY

The terms “invention,” “the invention,” “this invention”, the “present technology” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

The present technology includes devices for forming an anastomosis between two lumens, for example a first lumen and a second lumen. In embodiments, the device may include a staple receiving assembly and a staple dispensing assembly. The staple receiving assembly may include a first body defining a bottom surface surrounding a first central opening and a plurality of staple receiving cavities positioned around the first central opening. The staple dispensing assembly may include a second body defining a top surface surrounding a second central opening and a plurality of staple dispensing cavities positioned around the second central opening. The device may also include a plurality of linear staples positioned within the plurality of staple dispensing cavities. The staple receiving assembly and the staple dispensing assembly may be designed to retain everted ends of the first and second lumens between the bottom surface and the top surface. Further, the staple dispensing assembly may be designed to propel the plurality of linear staples from the plurality of staple dispensing cavities through the retained everted ends of the first and second lumens toward the plurality of staple receiving cavities in order to form an anastomosis between the first lumen and the second lumen.

In some embodiments, the first body may define a first gap connected to the first central opening to define a first open loop and the second body may define a second gap connected to the second central opening to define a second open loop. The first and second gaps may be designed so that after the anastomosis is formed the first lumen is removable from the first central opening through the first gap and the second lumen is removable from the second central opening through the second gap. In some embodiments, the first central opening and the second central opening are circular. In some embodiments, the first body defines a first internal vacuum passage fluidically coupled to a plurality of first suction openings on the bottom surface designed to be coupled to a vacuum source in order to retain the everted end of the first lumen to the bottom surface, and the second body defines a second internal vacuum passage fluidically coupled to a plurality of second suction openings on the top surface designed to be coupled to the vacuum source in order to retain the everted end of the second lumen to the top surface. The plurality of first suction openings may be radially positioned around the first central opening on the bottom surface, and the plurality of second suction openings may be radially positioned around the second central opening on the top surface.

In embodiments, the linear staples may each include a piercing end and a tail end on opposite sides of a longitudinal axis of the linear staple. The linear staples may each further include a first set of barbs between the piercing end and the tail end, and a second set of barbs between the first set of bards and the tail end. Each linear staple may be designed to retain the everted end of the first lumen between the first set of barbs and the second set of barbs, and be designed to retain the everted end of the second lumen between the second set of barbs and the tail end.

In embodiments, the second body may define an internal pressurized air passage fluidically coupled to the plurality of staple dispensing cavities and designed to be coupled to a pressurized air source in order to pneumatically propel the staples out of the plurality of staple dispensing cavities toward the plurality of staple receiving cavities.

In embodiments, devices may also include a hinge coupled to the staple receiving assembly and the staple dispensing assembly. The hinge may be designed to allow the device to transition between an open configuration wherein the staple receiving assembly and the staple dispensing assembly are spaced apart, and a closed configuration wherein the staple receiving assembly and the staple dispensing assembly retain the everted ends of the first and second lumens between the bottom surface and the top surface and the plurality of staple dispensing cavities are aligned with the plurality of staple receiving cavities.

In embodiments, the staple receiving assembly includes a plurality of first magnets arranged around the first central opening. And the staple dispensing assembly includes a plurality of second magnets arranged around the first central opening. The first and second magnets may be arranged in order to be attracted and cause the plurality of staple dispensing cavities to be aligned with the plurality of staple receiving cavities.

In embodiments, the devices may be used in a method for forming an anastomosis between a first lumen and a second lumen. The devices may include a staple receiving assembly including a first body defining a bottom surface surrounding a first central opening and a plurality of staple receiving cavities positioned around the first central opening, a staple dispensing assembly including a second body defining a top surface surrounding a second central opening and a plurality of staple dispensing cavities positioned around the second central opening, and a plurality of linear staples positioned within the plurality of staple dispensing cavities. The method may include a step of positioning a first end of the first lumen within the first central opening and everting the first end onto the bottom surface. The method may also include a step of positioning a second end of the second lumen within the second central opening and everting the second end onto the top surface. The method may also include a step of retaining the first end and the second end between the top surface and the bottom surface. The method may also include a step of propelling the plurality of linear staples from the plurality of staple dispensing cavities through the retained everted ends of the first and second lumens toward the plurality of staple receiving cavities in order to form an anastomosis between the first lumen and the second lumen.

In embodiments, the first body may define a first gap connected to the first central opening to define a first open loop, and the second body may define a second gap connected to the second central opening to define a second open loop. The method may include the step of removing the first lumen from the first central opening through the first gap and removing the second lumen from the second central opening through the second gap after the anastomosis is formed. The first central opening and the second central opening may be circular. The first body may define a first internal vacuum passage fluidically coupled to a plurality of first suction openings on the bottom surface, and the second body may define a second internal vacuum passage fluidically coupled to a plurality of second suction openings on the top surface. Methods may include the step of coupling a vacuum source to the first plurality of suction openings in order to evert the first end of the first lumen to the bottom surface. Methods may also include the step of coupling the vacuum source to the second plurality of suction openings in order to evert the second end of the second lumen to the top surface.

In embodiments, the plurality of first suction openings may be radially positioned around the first central opening on the bottom surface, and the plurality of second suction openings may be radially positioned around the second central opening on the top surface.

In embodiments, the methods may include the linear staples each including a piercing end and a tail end on opposite sides of a longitudinal axis of the linear staple. The linear staples may each further include a first set of barbs between the piercing end and the tail end, and a second set of barbs between the first set of bards and the tail end. In some embodiments, the method step of propelling the plurality of linear staples includes piercing the first end and the second end with the piercing end. In some embodiments, the anastomosis between the first lumen and the second lumen is formed by each linear staple retaining the everted first end of the first lumen between the first set of barbs and the second set of barbs, and retaining the everted second end of the second lumen between the second set of barbs and the tail end.

In embodiments, the second body defines an internal pressurized air passage fluidically coupled to the plurality of staple dispensing cavities, and the method further includes coupling the internal pressurized air passage to a pressurized air source in order to pneumatically propel the staples out of the plurality of staple dispensing cavities toward the plurality of staple receiving cavities.

In embodiments, the device further includes a hinge coupled to the staple receiving assembly and the staple dispensing assembly, and the method includes transitioning the device with the hinge from an open configuration wherein the staple receiving assembly and the staple dispensing assembly are spaced apart, to a closed configuration wherein the staple receiving assembly and the staple dispensing assembly retain the everted first end and second end of the first and second lumens between the bottom surface and the top surface and the plurality of staple dispensing cavities are aligned with the plurality of staple receiving cavities.

In embodiments, the first lumen and the second lumen are blood vessels.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIGS. 1A and 1B show a prior art method of hand sewing lumens to form an anastomosis.

FIGS. 2A-2C show a prior art device for forming an anastomosis.

FIGS. 3A and 3B show a device for forming an anastomosis according to embodiments of the present technology.

FIGS. 4A-4C show a staple for forming an anastomosis according to embodiments of the present technology.

FIG. 5 shows a staple receiving assembly for forming an anastomosis according to embodiments of the present technology.

FIGS. 6A and 6B show a staple dispensing assembly for forming an anastomosis according to embodiments of the present technology.

FIGS. 7A-7E show steps for forming an anastomosis according to embodiments of the present technology.

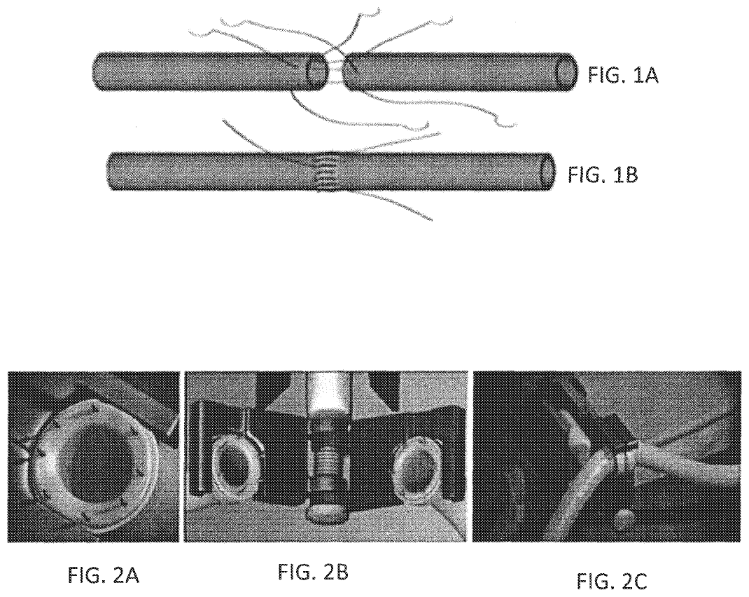

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described. Directional references such as “up,” “down,” “top,” “left,” “right,” “front,” and “back,” among others are intended to refer to the orientation as illustrated and described in the figure (or figures) to which the components and directions are referencing.

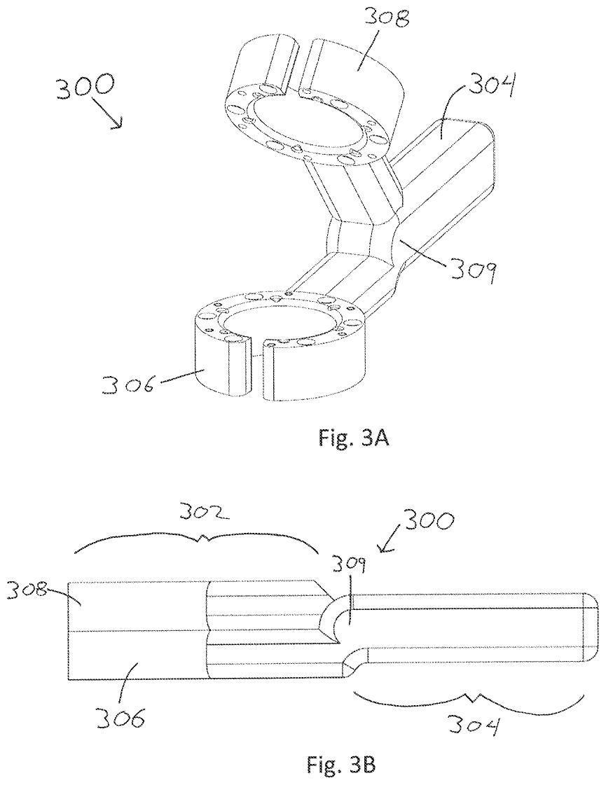

The present technology relates to devices 300 for forming anastomoses between two portions of material, for example forming anastomoses between two lumens. FIGS. 3A and 3B show views of an embodiment of a device 300, which may be used, for example, for forming anastomoses by applying anastomotic staples 310. The device 300 may include an applicator end 302 and a handle end 304. The handle end 304 may be shaped and sized to be manipulated by a user, for example a surgeon or a surgical robot, to form an anastomosis between two lumens, for example, two blood vessels of a patient, with the applicator end 302. While some of the examples described herein may relate to forming anastomoses in biological lumens, the devices and methods disclosed herein may be used for forming anastomoses in non-biological lumens, for example, tubing in an industrial setting.

The applicator end 302 may include a staple dispensing assembly 306 and a staple receiving assembly 308. The staple dispensing assembly 306 and staple receiving assembly 308 may be attached together with a hinge 309 so that the staple dispensing assembly 306 and staple receiving assembly 308 may pivot relative to each other between an open configuration wherein the staple dispensing assembly 306 and the staple receiving assembly 308 are spaced apart from each other, as shown, for example, in FIG. 3A, and a closed configuration, wherein the staple dispensing assembly 306 and the staple receiving assembly 308 are proximate and/or contacting each other, as shown, for example, in FIG. 3B. As will be discussed in greater detail below, the staple dispensing assembly 306 and the staple receiving assembly 308 may be placed in the closed configuration in order to dispense staples 310 from the staple dispensing assembly 306 in order to form an anastomosis. The spacing between the staple dispensing assembly 306 and the staple receiving assembly 308 in the closed configuration may correspond to the combined thicknesses of the portions of material, e.g. the combined thicknesses of the wall of the lumens, through which the staples 310 are applied to form the anastomosis.

In some embodiments, the staple dispensing assembly 306 and staple receiving assembly 308 may be detachable from the handle end 304 so that a set of the staple dispensing assembly 306 and the staple receiving assembly 308 previously used to form an anastomosis may be removed from the device 300, e.g. for disposal and/or sanitization (e.g. autoclaved), and replaced with a new set of a staple dispensing assembly 306 and a staple receiving assembly 308. This allows the handle end 304 to be reused during the forming of multiple anastomoses. The staple dispensing assembly 306 and staple receiving assembly 308 may be separately detachable or may be detachable as an assembly of both the staple dispensing assembly 306 and the staple receiving assembly 308 from the hinge 309 and/or the handle end 304.

In some embodiments, the staples 310 dispensed from the staple dispensing assembly 306 may be linear staples, in that the staples 310 extend along a substantially linear longitudinal axis that does not include substantial curvature or bends. In some embodiments, unlike conventional surgical staples which are deformed during application in order to create a mechanical locking shape or clamping force, for example by bending the staples from a first non-linear shape to a second non-linear shape, the linear staples 310 as disclosed herein may maintain substantially the same non-deformed shape prior to, during and after application.

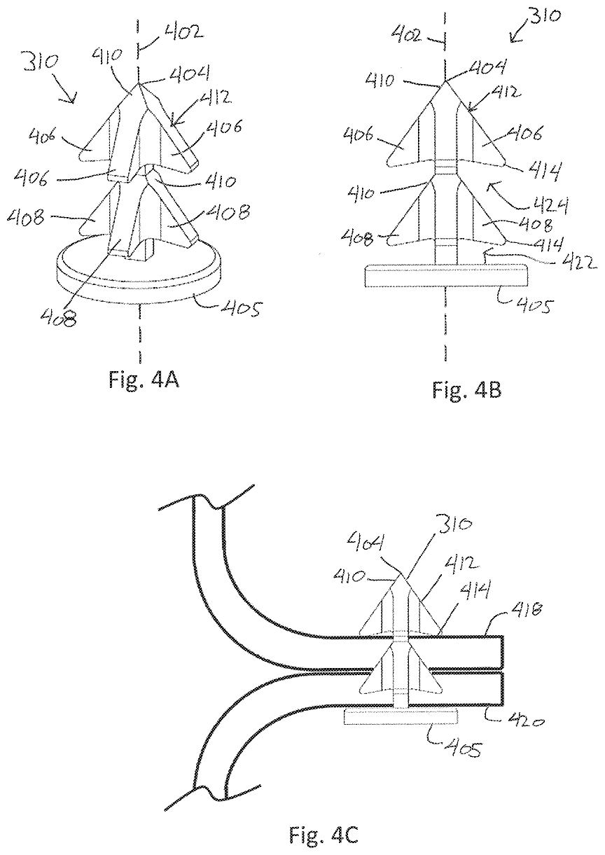

FIGS. 4A and 4B show views of a substantially linearly shaped staple 310 according to embodiments of the present technology. As shown, the staple 310 may extend along a straight longitudinal axis 402 from a piercing end 404 to a tail end 405. The staple 310 may be used to couple a first portion of material 418 to a second portion of material 420, for example the perimeter of an end of a first lumen to the perimeter of an end of second lumen. As used herein, the terms first portion of material and second portion of material does not convey an layering order, position, configuration, and/or method order, and the terms may be swapped with each other. Each portion of material may define a thickness through which the staple 310 extends. As shown, the staple 310 may include a first set of one or more barbs 406 proximate to the piercing end 404, and a second set of one or more barbs 408 proximate to the tail end 405. As shown in FIG. 4A, the profile of each set of barbs 406 and 408 may include a narrower portion 410 more proximate to the piercing end 404. An angled edge 412 of each barb may extend from the narrower portion 410 distally toward the tail end 304 to a wider portion 414, wherein at the wider portion 414 the profile of the barb may include an undercut portion 416 extending back toward the longitudinal axis 402. The angled edge 412 and undercut portion 416 of the profile of each barb allow for the staple 310 to be inserted into a portion of material in a first direction along the longitudinal axis 402 with the piercing end 404 of each bard leading, and be prevented from being easily extracted in a second direction, opposite the first direction, along the longitudinal axis 402 with the tail end 405 leading.

In some embodiments, the tail end 405 may extend radially from the longitudinal axis 402. The tail end 405 may extend the same distance or greater radially from longitudinal axis 402 relative to the first and second set of barbs 406 and 408. The tail end 405 may be circular in shape. The tail end 405 may be spaced apart from the undercut portion 416 of the second set of barbs 408. The staple 310 may define a first undercut recess 424 between the undercut portion 416 of the first set of barbs 406 and the angle edge 412 of the second set of barbs 408 for retaining the first portion of material 418, as shown in FIGS. 4B and 4C.

The staple 310 may also define a second undercut recess 422 between the undercut portion 416 of the second set of barbs 408 and the tail end 405 for retaining the second portion of material 420, as shown in FIGS. 4B and 4C. To couple the first portion of material 418 to the second portion of material 420, the first and second portions of material may be overlapped so that parallel inner surfaces contact, and parallel outer surfaces face away from each other. The piercing end 404 of the staple 310 may form an opening into the outer surface of the second portion of material 420 and the piercing end 404 and first set of barbs 406 may pass through the second portion of material 420. The piercing end 404 of the staple 310 may then exit the inner surface of the second portion of material 420, and then the piercing end 404 and the first set of barbs 406 may pass through the first portion of material 418 as the second set of barbs 408 passes through the second portion of material 420. The staple 310 may be advanced through the overlapping first and second portions of materials until the piercing end 404 exits the outer surface of the first portion of material 418, the tail end 405 contacts the outer surface of the second portion of material 420, the first portion material 418 is retained within the first undercut recesses 424, and the second portion of material is retained within the second undercut recesses 422, as shown in FIGS. 4B and 4C.

As shown in FIG. 4A, the first set and second of barbs 406 and 408 may each comprise a plurality of radially extending triangular barbs. In some embodiments, the plurality of barbs in the first set may include any number of barbs, for example, four barbs equally spaced in the radial direction, as shown in FIG. 4A. In some embodiments, each of the sets of barbs may include the same number of barbs and may be radially positioned in the same radial position in order to limit damage to the first portion of material, which both sets of barbs pass through during insertion of the staple 310. In some embodiments, a staple 310 may include any number of barbs and spacing of the first and second sets of barbs. In some embodiments, the one or more barbs of each set of barbs may include a single frusto-conical shaped barb.

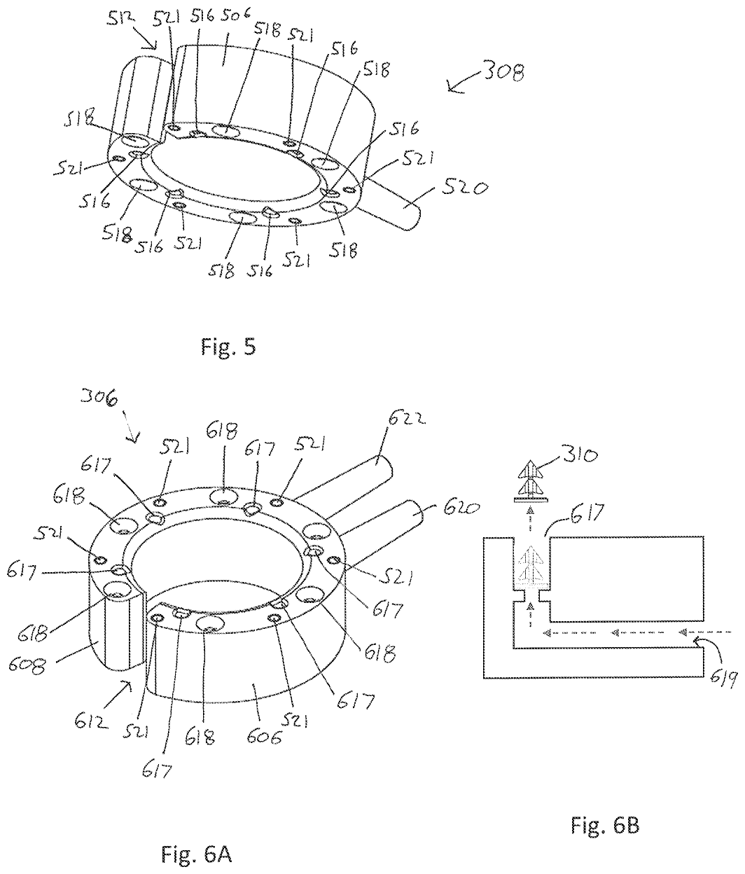

FIG. 5 shows an embodiment of a staple receiving assembly 308. As shown, a staple receiving assembly 308 may include a first body 502 defining an open loop. In some embodiments, the first body 502 is substantially circular defining an open loop circular central opening 504. The first body 502 may include a first left half loop 506 and first right half loop 508, each forming a portion of the open loop. The first left and right half loops 506 and 508 are coupled together at a proximal end 510 of the first body 502, and are separated by a gap 512 at a distal end of the first body 502. The central opening 504 is sized and shaped to correspond to an expanded configuration of a lumen to which the anastomoses is performed. For example the central opening 504 may be circular with a diameter between 2 mm and 10 mm and correspond to the size and shape of a blood vessel. The gap 512 is sized and shaped to correspond to a compressed configuration of the lumen to which anastomoses is performed so that prior to forming an anastomosis the lumen may be compressed to position the lumen within the central opening, and after forming the anastomosis the lumen may be compressed to pass through the gap 512 to remove the lumen from the central opening. In embodiments, the gap 512 is sized between 1 mm and 6 mm.

The first body 502 of the staple receiving assembly 308 defines a bottom surface 514. The bottom surface 514 may be planar and extends around a perimeter of the central opening 504. The first body 502 defines a plurality of staple receiving cavities 516, into which the piercing ends 404 of staples 310 are received when coupling a first portion of material to a second portion of material. The portion of bottom surface 514 surrounding each staple receiving cavity 516 provides backing support for the outside surface of the first portion of material during application of the staple 310.

The first body 502 of the staple receiving assembly 308 further defines an internal vacuum passage fluidically coupled to a plurality of suction openings 518 defined on the bottom surface of the first body 502, and a vacuum port 520. The plurality of suction openings 518 may be positioned between the staple receiving cavities 516. During use, the vacuum port 520 is coupled to a vacuum source, for example the vacuum source available in an operating room, in order to cause the first portion of material to be retained against the bottom surface 514 of the first body 502 due to the difference in pressure between the vacuum and atmosphere. Vacuum retention is beneficial in retaining the first portion of material in that the material is not damaged, and may be easily repositioned by releasing the vacuum and reapplying the vacuum. In some embodiments, the device 300 includes a vacuum control selector, e.g. a knob on the handle end, for controlling the vacuum amount and therefore the pressure for retaining the first portion of material.

As shown in FIG. 5, the vacuum port 520 may extend from the proximal end 510 of the first body 502. The vacuum port 520 may comprise an elongated tube for coupling to the handle end 304, the hinge 309, and/or directly to a vacuum line. In some embodiments, the vacuum port 520 may act as both a fluidic coupling and a mechanical coupling for mechanically coupling the staple receiving assembly 308 to other components of the device 300.

FIG. 6A shows an embodiment of a staple dispensing assembly 306. As shown, a staple dispensing assembly 306 may include a second body 602 defining an open loop. The second body 602 of the staple dispensing assembly 306 and the staple receiving assembly 308 may be complementary in shape. In some embodiments, the second body 602 is substantially circular defining an open loop circular central opening 604. The second body 602 includes a second left half loop 606 and a second right half loop 608. The second left and right half loops 606 and 608 are coupled at a proximal end 610 of the second body 602, and are separated by a gap 612 at a distal end of the second body 602. The second central opening 604 is sized and shaped to correspond to an expanded configuration of a lumen to which the anastomoses formed with. The 612 gap is sized and shaped to correspond to a compressed configuration of the lumen to which anastomoses is formed so that prior to forming the anastomoses the lumen may be compressed to position the lumen within the central opening 604, and after anastomose the lumen may be compressed to pass through the gap 612 to remove the lumen from the central opening 604.

The second body 602 of the staple dispensing assembly 306 defines a top surface 614. The top surface 614 may be planar and extends around a perimeter of the central opening 604. The second body 602 define a plurality of staple dispensing cavities 617, out of which the staples 310 are dispensed with the piercing ends 404 of the staples 310 pointing out of the staple dispensing cavities 617 when coupling a first portion of material to a second portion of material. The top surface 614 is sized and shaped to correspond to the bottom surface 514 of the first body 502 of the staple receiving assembly. 308 As will be discussed in greater detail below, the overlapped first portion of material and second portion of material are retained between the top surface 614 and the bottom surface 514.

The plurality of staple dispensing cavities 617 are spaced radially around top surface 614 and are positioned to correspond with complementary staple receiving cavities 516 of the first body 502 of the staple receiving assembly 308. The plurality of staple dispensing cavities 617, and corresponding staple receiving cavities 516, may be uniformly spaced in a circle around the central openings 504 and 604, including the spacing of the staple receiving cavities 516 and staple dispensing cavities 617 of opposing sides of the gaps 512 and 612. In some embodiments, the hinge 309 maintains alignment of the staple dispensing assembly 306 and the staple receiving assembly 308 so that in the closed configuration, the staple dispensing cavities 617 overlap with the staple receiving cavities 516. In some embodiments, the staple dispensing assembly 306 and the staple receiving assembly 308 each include magnets 521 complementarily positioned so that in the closed configuration the attraction of the complementary magnets causes the staple dispensing cavities 617 to align with the staple receiving cavities 516.

The second body 602 of the staple dispensing assembly 306 further defines an internal vacuum passage fluidically coupled to a plurality of suction openings 618 defined on the top surface 614 of the second body 602, and a vacuum port 620. The plurality of suction openings 618 may be positioned between the staple dispensing cavities 617. During use, the vacuum port 620 is coupled to a vacuum source, for example the vacuum source available in an operating room, in order to cause the second portion of material to be retained against the top surface 614 of the second body 602 due to the difference in pressure between the vacuum and atmosphere. Vacuum retention is beneficial in retaining the second portion of material in that the material is not damaged, and may be easily repositioned by releasing the vacuum and reapplying the vacuum. As noted above, in some embodiments, the device 300 may include a vacuum control selector. The vacuum control selector may allow for individual and/or combined control of the vacuum amount of the first plurality of suction openings 518 and the second plurality of suction openings 618.

In some embodiments, for example as shown in FIG. 6B, the staples 310 may be pneumatically propelled out of the staple dispensing cavities 617. As shown in FIG. 6B, the second body 602 of the staple dispensing assembly 306 may further defines an internal pressurized air passage 619 fluidically coupled to the plurality of staple dispensing cavities 617 and a pressurized air port 622. The tail end 405 of the staples 310 may correspond in shape and size to the cross-section of the staple dispensing cavities 617 in order to form a seal allowing the pressurized air to propel the staple 310 out of the staple dispensing cavities 617. For example, as shown the tail end 405 and cross-section of the staple dispensing cavities 617 may be may be circular. In some embodiments, the staple dispensing assembly 306 may include pneumatically actuated captive pistons within each staple dispensing cavity 617 which push the staples 310 out of the staple dispensing cavity 617. In some embodiments, the staple dispensing assembly 306 may include mechanically actuated captive pistons within each staple delivery cavity which push the staples out of the staple delivery cavities.

During use, the pressurized air port 622 may be coupled to a pressurized air source, for example the pressurized air source available in an operating room. As shown in FIG. 6A, the pressurized air port 622 and vacuum port 620 may extend from the proximal end of the second body 602. The pressurized air port 622 and vacuum port 620 may each comprise an elongated tube for coupling to the handle end 304, the hinge 309, and/or directly to respective lines connected to the pressurized air source and vacuum source. In some embodiments, the air port 622 and vacuum port 620 may act as both fluidic couplings and a mechanical coupling for mechanically coupling the staple dispensing assembly 306 to other components of the device 300.

In some embodiments, a device 300 may include two staple dispensing/receiving assemblies including the features of both the staple dispensing assemblies 306 and staple receiving assemblies 308, as disclosed above. In such embodiments, staples 310 may be delivered in opposite directions to form an anastomosis.

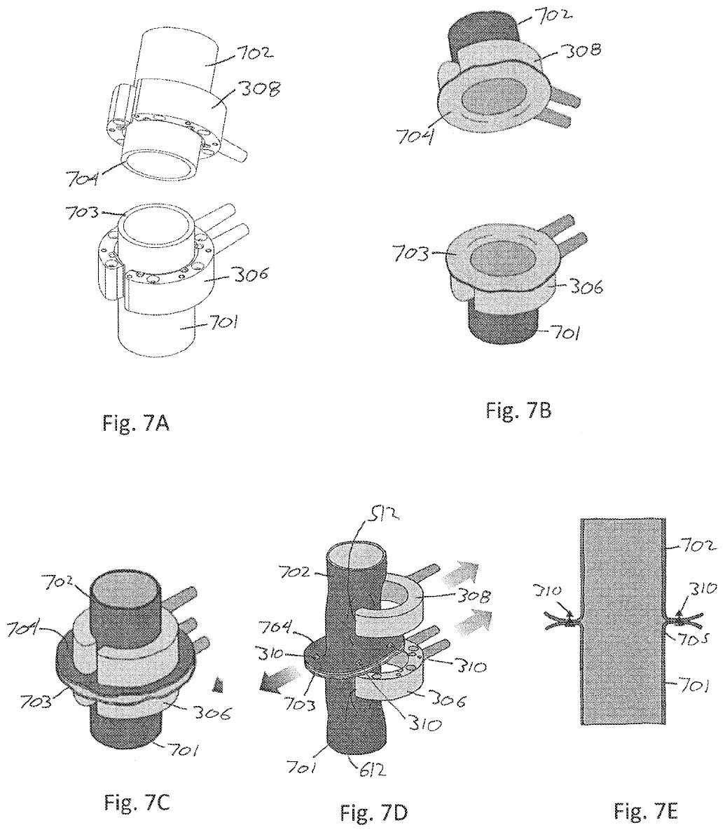

FIGS. 7A-7E show steps for using a device 300 as disclosed herein for forming an anastomosis between a first lumen 701 and a second lumen 702. As show in FIG. 7A the first and second lumens 701 and 702 may be positioned in the respective central openings 504 and 604 of the first body 502 and the second body 602 of the respective staple receiving assembly 308 and staple dispensing assembly 306. In some embodiments, the first and second lumen 701 and 702 may be positioned in the central openings 504 and 604 by translating the lumen longitudinally through a central axis of the central openings 504 and 604. For example, in methods related to vascular anastomoses, vascular forceps may be used to bring the end of each vessel through the central openings. In some embodiments, the first and second lumen 701 and 702 may be position in the central openings 504 and 604 by compressing the lumens and passing the compressed lumens into the central openings 504 and 604 through the gaps 512 and 612. As shown in FIG. 7A the lumens 701 and 702 may be positioned so than the ends 703 and 704 are positioned a distance from the respective top surface 614 and bottom surface 514. The distance may correspond to a width of the top surface 614 and bottom surface 514 so the ends 703 and 704 of the lumens 701 and 702 may be everted as shown in FIG. 7B. A user may manipulate the ends 703 and 704 of the lumens 701 and 702 in an everted configuration, and the suction openings 518, as disclosed above, may draw the ends toward the top and bottom surfaces 514 and 614 and retain the ends 703 and 704 of the lumens 701 and 702 with the device in the opened configuration, as shown in FIG. 7B. Due to the radially suction force the process of everting and retaining the ends of the lumens may be performed quickly, for example in less than a minute. In methods corresponding to vascular anastomoses, ends of the lumens are vessel ends and are flared like a trumpet horn with the endothelium of each vessel end facing each other, ready for apposition.

With the ends 703 and 704 of the lumens 701 and 702 retained against the top surface 614 and bottom surface 514 in the everted configuration and with the device 300 in the open configuration, the device 300 may be transitioned to the closed configuration, as shown in FIG. 7C. In the closed configuration, the end 703 of the first lumen 701 and the end 704 of the second lumen 702 are overlapped and contacting. In the configuration shown in FIG. 7C the first lumen and the second lumen are aligned in order to form the anastomosis.

With the first lumen 701 and second lumen 702 aligned and each end 703 and 704 everted, the staple dispensing assembly 306 may be actuated in order to propel the staples 310 through the end 703 of the first lumen 701. As noted above, the staples 310 may be pneumatically propelled into the material, and the bottom surface 514 of the staple receiving assembly 308 may act as a backing to support the overlapped material so that the staples may pierce through both portions of material with the piercing ends 404 occupying a portion of the staple receiving cavities 516, and the tail ends 405 abutting the outer surface of the lower portion of material. In some embodiments, the staples 310 may be simultaneously propelled into the second and first portions of material. The number and or size of the staples delivered by a staple dispensing assembly may be based on the size of the lumen. For example as shown in FIG. 7D, six staples 310 are used to form the anastomosis.

With the staples 310 positioned within the ends 703 and 704 of the first and second lumens 701 and 702 the device 300 may be transitioned from the closed configuration to the open configuration. In some embodiments, prior to transitioning, retaining the first and second portions of material with the suction may be ceased. In the open configuration, the first and second lumens 701 and 702 may be compressed radially so that the coupled lumens may be removed through the gaps 512 and 612 of the staple receiving assembly 308 and staple dispensing assembly 306, as shown in FIG. 7D. During vascular anastomosis, the steps shown in FIGS. 7A-7D may be performed between 2 and 5 minutes due to the efficiency of the eversion with suction, and pneumatic dispensing of the staples.

With the anastomosis formed, as shown in FIG. 7E, the first lumen 701, second lumen 702, and anastomosis 705 therebetween are free to expand and contract due to no radial constriction imposed by the staples coupling the ends together. This ability to expand and contract is beneficial in vascular anastomosis in allowing expansion of the blood vessel with every heartbeat.

Other variations are within the spirit of the present disclosure. Thus, while the disclosed techniques are susceptible to various modifications and alternative constructions, certain illustrated examples thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims. For instance, any of the examples, alternative examples, etc., and the concepts thereof may be applied to any other examples described and/or within the spirit and scope of the disclosure.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. The phrase “based on” should be understood to be open-ended, and not limiting in any way, and is intended to be interpreted or otherwise read as “based at least in part on,” where appropriate. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate examples of the disclosure and does not pose a limitation on the scope of the disclosure unless otherwise claimed. No language in the specification should be construed as indicating any non-claimed element as essential to the practice of the disclosure.

1. A device for forming an anastomosis between a first lumen and a second lumen, the device comprising:
a staple receiving assembly comprising a first body defining a bottom surface surrounding a first central opening and a plurality of staple receiving cavities positioned around the first central opening;
a staple dispensing assembly comprising a second body defining a top surface surrounding a second central opening and a plurality of staple dispensing cavities positioned around the second central opening; and
a plurality of linear staples positioned within the plurality of staple dispensing cavities,
wherein the staple receiving assembly and the staple dispensing assembly are configured to retain everted ends of the first and second lumens between the bottom surface and the top surface, and
wherein the staple dispensing assembly is configured to propel the plurality of linear staples from the plurality of staple dispensing cavities through the retained everted ends of the first and second lumens toward the plurality of staple receiving cavities in order to form an anastomosis between the first lumen and the second lumen.

2. The device of claim 1, wherein the first body defines a first gap connected to the first central opening to define a first open loop,
wherein the second body defines a second gap connected to the second central opening to define a second open loop, and
wherein the first and second gaps are configured so that after the anastomosis is formed the first lumen is removable from the first central opening through the first gap and the second lumen is removable from the second central opening through the second gap.

3. The device of claim 2, wherein the first central opening and the second central opening are circular.

4. The device of claim 1, wherein the first body defines a first internal vacuum passage fluidically coupled to a plurality of first suction openings on the bottom surface configured to be coupled to a vacuum source in order to retain the everted end of the first lumen to the bottom surface, and
wherein the second body defines a second internal vacuum passage fluidically coupled to a plurality of second suction openings on the top surface configured to be coupled to the vacuum source in order to retain the everted end of the second lumen to the top surface.

5. The device of claim 4, wherein the plurality of first suction openings are radially positioned around the first central opening on the bottom surface, and
wherein the plurality of second suction openings are radially positioned around the second central opening on the top surface.

6. The device of claim 1, wherein the linear staples each comprise a piercing end and a tail end on opposite sides of a longitudinal axis of the linear staple, and
wherein the linear staples each further comprises a first set of barbs between the piercing end and the tail end, and a second set of barbs between the first set of bards and the tail end.

7. The device of claim 6, wherein each linear staple is configured to retain the everted end of the first lumen between the first set of barbs and the second set of barbs, and to retain the everted end of the second lumen between the second set of barbs and the tail end.

8. The device of claim 1, wherein the second body defines an internal pressurized air passage fluidically coupled to the plurality of staple dispensing cavities and configured to be coupled to a pressurized air source in order to pneumatically propel the staples out of the plurality of staple dispensing cavities toward the plurality of staple receiving cavities.

9. The device of claim 1, further comprising a hinge coupled to the staple receiving assembly and the staple dispensing assembly, and
wherein the hinge is configured to allow the device to transition between an open configuration wherein the staple receiving assembly and the staple dispensing assembly are spaced apart, and a closed configuration wherein the staple receiving assembly and the staple dispensing assembly retain the everted ends of the first and second lumens between the bottom surface and the top surface and the plurality of staple dispensing cavities are aligned with the plurality of staple receiving cavities.

10. The device of claim 1, wherein the staple receiving assembly comprises a plurality of first magnets arranged around the first central opening,
wherein the staple dispensing assembly comprises a plurality of second magnets arranged around the first central opening, and
wherein the first and second magnets are arranged in order to be attracted and cause the plurality of staple dispensing cavities to be aligned with the plurality of staple receiving cavities.

11. A method for forming an anastomosis between a first lumen and a second lumen with a device comprising a staple receiving assembly comprising a first body defining a bottom surface surrounding a first central opening and a plurality of staple receiving cavities positioned around the first central opening, a staple dispensing assembly comprising a second body defining a top surface surrounding a second central opening and a plurality of staple dispensing cavities positioned around the second central opening, and a plurality of linear staples positioned within the plurality of staple dispensing cavities, the method comprising:
positioning a first end of the first lumen within the first central opening and everting the first end onto the bottom surface;
positioning a second end of the second lumen within the second central opening and everting the second end onto the top surface;
retaining the first end and the second end between the top surface and the bottom surface; and
propelling the plurality of linear staples from the plurality of staple dispensing cavities through the retained everted ends of the first and second lumens toward the plurality of staple receiving cavities in order to form an anastomosis between the first lumen and the second lumen.

12. The method of claim 11, wherein the first body defines a first gap connected to the first central opening to define a first open loop,
wherein the second body defines a second gap connected to the second central opening to define a second open loop, and
wherein the method further comprises:
removing the first lumen from the first central opening through the first gap and removing the second lumen from the second central opening through the second gap after the anastomosis is formed.

13. The method of claim 12, wherein the first central opening and the second central opening are circular.

14. The method of claim 13, wherein the first body defines a first internal vacuum passage fluidically coupled to a plurality of first suction openings on the bottom surface, wherein the second body defines a second internal vacuum passage fluidically coupled to a plurality of second suction openings on the top surface, and
wherein the method further comprises:
coupling a vacuum source to the first plurality of suction openings in order to evert the first end of the first lumen to the bottom surface, and
coupling the vacuum source to the second plurality of suction openings in order to evert the second end of the second lumen to the top surface.

15. The method of claim 14, wherein the plurality of first suction openings are radially positioned around the first central opening on the bottom surface, and
wherein the plurality of second suction openings are radially positioned around the second central opening on the top surface.

16. The method of claim 11, wherein the linear staples each comprise a piercing end and a tail end on opposite sides of a longitudinal axis of the linear staple,
wherein the linear staples each further comprises a first set of barbs between the piercing end and the tail end, and a second set of barbs between the first set of bards and the tail end, and
wherein propelling the plurality of linear staples comprises piercing the first end and the second end with the piercing end.

17. The method of claim 16, wherein the anastomosis between the first lumen and the second lumen is formed by each linear staple retaining the everted first end of the first lumen between the first set of barbs and the second set of barbs, and retaining the everted second end of the second lumen between the second set of barbs and the tail end.

18. The method of claim 11, wherein the second body defines an internal pressurized air passage fluidically coupled to the plurality of staple dispensing cavities,
wherein the method further comprises coupling the internal pressurized air passage to a pressurized air source in order to pneumatically propel the staples out of the plurality of staple dispensing cavities toward the plurality of staple receiving cavities.

19. The method of claim 11, wherein the device further comprises a hinge coupled to the staple receiving assembly and the staple dispensing assembly, and
wherein the method comprises transitioning the device with the hinge from an open configuration wherein the staple receiving assembly and the staple dispensing assembly are spaced apart, to a closed configuration wherein the staple receiving assembly and the staple dispensing assembly retain the everted first end and second end of the first and second lumens between the bottom surface and the top surface and the plurality of staple dispensing cavities are aligned with the plurality of staple receiving cavities.

20. The method of claim 11, wherein the staple receiving assembly comprises a plurality of first magnets arranged around the first central opening,
wherein the staple dispensing assembly comprises a plurality of second magnets arranged around the first central opening, and
wherein the first and second magnets are arranged in order to be attracted and cause the plurality of staple dispensing cavities to be aligned with the plurality of staple receiving cavities.

21. The method of claim 11, wherein the first lumen and the second lumen are blood vessels.
